(19) United States
(12) Patent Application Publication
(10) Pub. No.: US 2024/0401141 A1
(43) Pub. Date: Dec. 5, 2024

(54) METHODS AND MATERIALS FOR IDENTIFYING AND TREATING TRAUMATIC BRAIN INJURY

(71) Applicant: Mayo Foundation for Medical Education and Research, Rochester, MN (US)

(72) Inventors: Ross C. Puffer, Rochester, MN (US); Ian F. Parney, Rochester, MN (US)

(73) Assignee: Mayo Foundation for Medical Education and Research, Rochester, MN (US)

(21) Appl. No.: 18/804,293

(22) Filed: Aug. 14, 2024

Related U.S. Application Data
(62) Division of application No. 17/150,926, filed on Jan. 15, 2021, now Pat. No. 12,091,716.
(60) Provisional application No. 62/961,343, filed on Jan. 15, 2020.

(51) Int. Cl. C12Q 1/6883

(52) U.S. Cl. CPC C12Q 1/6883; C12Q 2600/112; C12Q 2600/178

(57) ABSTRACT

This document provides methods and materials involved in identifying mammals as having, or being likely to have, a traumatic brain injury (TBI). This document also provides methods for treating mammals identified as having, or being likely to have, a TBI, based on the identification methods described herein.

### CROSS-REFERENCE TO RELATED APPLICATIONS

This application is a divisional of U.S. Utility application Ser. No. 17/150,926, filed Jan. 15, 2021, which claims the benefit of U.S. Provisional Application Ser. No. 62/961,343, filed Jan. 15, 2020. The disclosures of the prior applications are considered part of (and are incorporated by reference in) the disclosure of this application.

### SEQUENCE LISTING

This application contains a Sequence Listing that has been submitted electronically as an XML file named “07039-1946002_ST26_SL.XML.” The XML file, created on Aug. 9, 2024, is 11,558 bytes in size. The material in the XML file is hereby incorporated by reference in its entirety.

### TECHNICAL FIELD

This document relates to methods and materials for identifying and treating traumatic brain injury.

### BACKGROUND

Traumatic brain injury (TBI) is a leading cause of death in men under 35, and is increasingly recognized as a contributor to chronic neurological dysfunction. Plasma biomarkers such as glial fibrillary acidic protein (GFAP) in TBI have been identified.

### SUMMARY

This document is based, at least in part, on the identification of extracellular vesicles (EVs) and their contents as biomarkers of TBI. EVs are nanoscale membrane-encapsulated particles released by cells. Plasma EVs have been utilized as a source of biomarkers for cancer, but have not been extensively evaluated in other pathologies. As described herein, EVs can be isolated from peripheral blood after TBI, and brain-specific biomarkers contained therein can be analyzed and used for diagnosis, assessing prognosis, and monitoring treatment of TBI patients. The methods provided herein can be particularly useful, in some cases, in the emergency department for detecting evidence of TBI in cases where a computed tomography (CT) scan may be negative. Given that patients can develop post-TBI brain dysfunction even with a negative CT scan, identifying these cases may be important to guide diagnosis and/or treatment. These methods may also be relevant to diagnosing a concussion in the early phase of injury, as well as tracking recovery in the following days/weeks.

This document provides methods and materials involved in identifying mammals with TBI (e.g., humans with TBI). For example, this document provides methods and materials for detecting the presence of EVs containing an altered level of one or more microRNAs (miRNAs) (e.g., hsa-miR-1-3p, hsa-miR-143-3p, hsa-miR-151b, hsa-miR-27a-3p, hsa-miR-29a-3p, hsa-miR-328-3p, hsa-miR-9-3p, hsa-miR-99a-5p, hsa-miR-99b-5p, hsa-miR-155-5p, and/or hsa-miR-30c-5p) in a biological sample (e.g., an EV-containing blood or serum sample) from a mammal, thereby identifying the mammal as being more likely to have a TBI than a mammal that does not exhibit an altered level of the one or more miRNAs or other markers. Conversely, this document provides methods and materials for determining that a biological sample from a mammal does not have an altered level of the one or more miRNAs or other markers, thereby identifying the mammal as being less likely to have a TBI than a mammal that does exhibit an altered level of the one or more miRNAs or other markers. Identifying mammals as having or not having an altered level of one or more of the markers listed herein can allow clinicians to proceed with proper clinical surveillance (e.g., frequency of clinical follow-ups and/or need for surveillance biopsies) and treatment options for potential TBI patients.

This document also provides methods and materials involved in treating mammals identified as having a TBI. These markers can provide valuable information with regard to ongoing brain injury after an initial TBI, and can guide treatment decisions such as blood pressure management, cerebrospinal fluid (CSF) diversion, or even invasive surgical decompression. In the post-treatment phase, the markers can assist with tracking the effectiveness of these or other directed treatments. For example, this document provides methods and materials for administering blood pressure management, CSF diversion, or invasive surgical decompression, based at least in part on the markers described herein. In addition, mammals identified as being likely to have a severe TBI can undergo more regular surveillance (e.g., examination on a more frequent basis) to assess symptoms or effects of the TBI.

In a first aspect, this document features a method for identifying a mammal as having a TBI or as being likely to have a TBI. The method can include identifying a mammal as having EVs with an altered level of one or more miRNAs as compared to the level of the one or more miRNAs in EVs from a corresponding mammal that does not have a TBI, where the one or more miRNAs are selected from the group consisting of hsa-miR-1-3p (SEQ ID NO:2), hsa-miR-143-3p (SEQ ID NO:3), hsa-miR-151b (SEQ ID NO: 4), hsa-miR-27a-3p (SEQ ID NO:5), hsa-miR-29a-3p (SEQ ID NO:6), hsa-miR-328-3p (SEQ ID NO:7), hsa-miR-9-3p (SEQ ID NO:8), hsa-miR-99a-5p (SEQ ID NO:9), hsa-miR-99b-5p (SEQ ID NO:10), hsa-miR-155-5p (SEQ ID NO:11), and hsa-miR-30c-5p (SEQ ID NO:12). The mammal can be a human. The EVs can be from a blood sample obtained from the mammal. The blood sample can be a plasma sample. The method can include identifying the mammal as having EVs with altered levels of two or more of the miRNAs, as compared to levels of the two or more miRNAs in EVs from a corresponding mammal that does not have a TBI. The method can include identifying the mammal as having EVs with an increased level of one or more miRNAs selected from the group consisting of hsa-miR-1-3p (SEQ ID NO:2), hsa-miR-143-3p (SEQ ID NO:3), hsa-miR-151b (SEQ ID NO:4), hsa-miR-27a-3p (SEQ ID NO:5), hsa-miR-29a-3p (SEQ ID NO: 6), hsa-miR-328-3p (SEQ ID NO:7), hsa-miR-9-3p (SEQ ID NO:8), hsa-miR-99a-5p (SEQ ID NO:9), and hsa-miR-99b-5p (SEQ ID NO:10), as compared to a corresponding mammal that does not have a TBI. The method can include identifying the mammal as having EVs with a decreased level of one or more miRNAs selected from the group consisting of hsa-miR-155-5p (SEQ ID NO:11) and hsa-miR-30c-5p (SEQ ID NO: 12), as compared to a corresponding mammal that does not have a TBI. The method can include identifying the mammal as having EVs with an increased level of one or more miRNAs selected from the group consisting of hsa-miR-1-3p (SEQ ID NO:2), hsa-miR-143-3p (SEQ ID NO:3), hsa-miR-151b (SEQ ID NO:4), hsa-miR-27a-3p (SEQ ID NO: 5), hsa-miR-29a-3p (SEQ ID NO:6), hsa-miR-328-3p (SEQ ID NO:7), hsa-miR-9-3p (SEQ ID NO:8), hsa-miR-99a-5p (SEQ ID NO:9), and hsa-miR-99b-5p (SEQ ID NO:10), and as having a decreased level of one or more miRNAs selected from the group consisting of hsa-miR-155-5p (SEQ ID NO:11) and hsa-miR-30c-5p (SEQ ID NO:12), as compared to a corresponding mammal that does not have a TBI.

In another aspect, this document features a method that includes (a) identifying a mammal as having EVs with an altered level of one or more miRNAs as compared to a level of the one or more miRNAs in EVs from a corresponding mammal that does not have a TBI, where the miRNAs are selected from the group consisting of hsa-miR-1-3p (SEQ ID NO:2), hsa-miR-143-3p (SEQ ID NO:3), hsa-miR-151b (SEQ ID NO:4), hsa-miR-27a-3p (SEQ ID NO:5), hsa-miR-29a-3p (SEQ ID NO:6), hsa-miR-328-3p (SEQ ID NO: 7), hsa-miR-9-3p (SEQ ID NO:8), hsa-miR-99a-5p (SEQ ID NO:9), hsa-miR-99b-5p (SEQ ID NO:10), hsa-miR-155-5p (SEQ ID NO:11), and hsa-miR-30c-5p (SEQ ID NO: 12), thereby identifying the mammal as having or being likely to have a TBI, and (b) treating the mammal by one or more of (i) administering a medication to manage blood pressure, cerebral perfusion pressure, or intracranial pressure, (ii) administering an antibiotic, (iii) surgically removing a mass or lesion from the brain, and (iv) decompressing the brain via direct craniectomy and bone removal, where the treating reduces the effects of a TBI in the mammal. The mammal can be a human. The EVs can be from a blood sample obtained from the mammal. The blood sample can be a plasma sample. The method can include identifying the mammal as having EVs with altered levels of two or more of the miRNAs, as compared to levels of the two or more miRNAs in EVs from a corresponding mammal that does not have a TBI. The method can include identifying the mammal as having EVs with an increased level of one or more miRNAs selected from the group consisting of hsa-miR-1-3p (SEQ ID NO:2), hsa-miR-143-3p (SEQ ID NO:3), hsa-miR-151b (SEQ ID NO:4), hsa-miR-27a-3p (SEQ ID NO:5), hsa-miR-29a-3p (SEQ ID NO:6), hsa-miR-328-3p (SEQ ID NO:7), hsa-miR-9-3p (SEQ ID NO: 8), hsa-miR-99a-5p (SEQ ID NO:9), and hsa-miR-99b-5p (SEQ ID NO:10), as compared to a corresponding mammal that does not have a TBI. The method can include identifying the mammal as having EVs with a decreased level of one or more miRNAs selected from the group consisting of hsa-miR-155-5p (SEQ ID NO:11) and hsa-miR-30c-5p (SEQ ID NO: 12), as compared to a corresponding mammal that does not have a TBI. The method can include identifying the mammal as having EVs with an increased level of one or more miRNAs selected from the group consisting of hsa-miR-1-3p (SEQ ID NO: 2), hsa-miR-143-3p (SEQ ID NO:3), hsa-miR-151b (SEQ ID NO:4), hsa-miR-27a-3p (SEQ ID NO:5), hsa-miR-29a-3p (SEQ ID NO:6), hsa-miR-328-3p (SEQ ID NO: 7), hsa-miR-9-3p (SEQ ID NO:8), hsa-miR-99a-5p (SEQ ID NO:9), and hsa-miR-99b-5p (SEQ ID NO:10), and as having a decreased level of one or more miRNAs selected from the group consisting of hsa-miR-155-5p (SEQ ID NO:11) and hsa-miR-30c-5p (SEQ ID NO:12), as compared to a corresponding mammal that does not have a TBI.

In another aspect, this document features a method for treating a mammal having a TBI, where the mammal was identified as having EVs with an altered level of one or more miRNAs as compared to a level of the one or more miRNAs in EVs from a corresponding mammal that does not have a TBI, where the one or more miRNAs are selected from the group consisting of hsa-miR-1-3p (SEQ ID NO:2), hsa-miR-143-3p (SEQ ID NO:3), hsa-miR-151b (SEQ ID NO:4), hsa-miR-27a-3p (SEQ ID NO:5), hsa-miR-29a-3p (SEQ ID NO:6), hsa-miR-328-3p (SEQ ID NO:7), hsa-miR-9-3p (SEQ ID NO: 8), hsa-miR-99a-5p (SEQ ID NO:9), hsa-miR-99b-5p (SEQ ID NO:10), hsa-miR-155-5p (SEQ ID NO:11), and hsa-miR-30c-5p (SEQ ID NO:12), and where the method includes treating the mammal by one or more of (a) administering a medication to manage blood pressure, cerebral perfusion pressure, or intracranial pressure, (b) administering an antibiotic, (c) surgically removing a mass or lesion from the brain, and (d) decompressing the brain via direct craniectomy and bone removal, where the treating reduces the effects of TBI in the mammal. The mammal can be a human. The EVs can be from a blood sample obtained from the mammal. The blood sample can be a plasma sample. The mammal can be identified as having EVs with altered levels of two or more of the miRNAs, as compared to the levels of the two or more miRNAs in EVs from a corresponding mammal that does not have a TBI. The mammal can be identified as having EVs with an increased level of one or more miRNAs selected from the group consisting of hsa-miR-1-3p (SEQ ID NO:2), hsa-miR-143-3p (SEQ ID NO:3), hsa-miR-151b (SEQ ID NO:4), hsa-miR-27a-3p (SEQ ID NO:5), hsa-miR-29a-3p (SEQ ID NO:6), hsa-miR-328-3p (SEQ ID NO:7), hsa-miR-9-3p (SEQ ID NO:8), hsa-miR-99a-5p (SEQ ID NO: 9), and hsa-miR-99b-5p (SEQ ID NO:10), as compared to a corresponding mammal that does not have a TBI. The mammal can be identified as having EVs with a decreased level of one or more miRNAs selected from the group consisting of hsa-miR-155-5p (SEQ ID NO:11) and hsa-miR-30c-5p (SEQ ID NO:12), as compared to a corresponding mammal that does not have a TBI. The mammal can be identified as having EVs with an increased level of one or more miRNAs selected from the group consisting of hsa-miR-1-3p (SEQ ID NO:2), hsa-miR-143-3p (SEQ ID NO:3), hsa-miR-151b (SEQ ID NO:4), hsa-miR-27a-3p (SEQ ID NO:5), hsa-miR-29a-3p (SEQ ID NO:6), hsa-miR-328-3p (SEQ ID NO:7), hsa-miR-9-3p (SEQ ID NO:8), hsa-miR-99a-5p (SEQ ID NO: 9), and hsa-miR-99b-5p (SEQ ID NO:10), and as having a decreased level of one or more miRNAs selected from the group consisting of hsa-miR-155-5p (SEQ ID NO:11) and hsa-miR-30c-5p (SEQ ID NO:12), as compared to a corresponding mammal that does not have a TBI.

In another aspect, this document features a method for clearing a mammal for normal activity, wherein the mammal had a TBI or is suspected of having a TBI. The method comprises (or consists essentially or consists of) (a) identifying the mammal as having EVs that comprise levels of hsa-miR-1-3p (SEQ ID NO:2), hsa-miR-143-3p (SEQ ID NO: 3), hsa-miR-151b (SEQ ID NO:4), hsa-miR-27a-3p (SEQ ID NO:5), hsa-miR-29a-3p (SEQ ID NO:6), hsa-miR-328-3p (SEQ ID NO:7), hsa-miR-9-3p (SEQ ID NO:8), hsa-miR-99a-5p (SEQ ID NO:9), hsa-miR-99b-5p (SEQ ID NO:10), hsa-miR-155-5p (SEQ ID NO:11), and hsa-miR-30c-5p (SEQ ID NO:12) that are less than 5 percent altered as compared to the levels of the miRNAs in EVs from a corresponding mammal that does not have a TBI, and (b) instructing the mammal to resume one or more normal activities. The mammal can be a human. The EVs can be from a blood sample obtained from the mammal. The blood sample can be a plasma sample. The method can comprise instructing the mammal to resume a sporting activity.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A-1E provide results from nanoparticle tracking analyses. This figure includes representative histograms (FIGS. 1A and 1B) and photomicrographs (FIGS. 1C and 1D) of EV analysis in a healthy control (FIGS. 1A and 1C) and a TBI patient (FIG. 1B and 1D). The histograms in both the healthy control (FIG. 1A) and TBI patient (FIG. 1B) demonstrate a substantial exosome peak (<100 nm) and multiple smaller microvesicle peaks (>100 nm). FIG. 1E is a western blot showing expression of common EV-associated proteins in EVs isolated from five donors with this protocol.

FIGS. 2A-2D show EV characteristics, with a comparison between TBI patients and normal donors with normal consciousness (Glasgow Coma Scale (GCS)=15) vs. TBI patients with altered consciousness (GCS<14) for EV concentration (FIG. 2A), EV size (FIG. 2B), EV GFAP yield (FIG. 2C), and EV RNA yield (FIG. 2D). It was notable that the GFAP yield was significantly higher in TBI patients with altered consciousness, but no significant differences were seen in EV concentration, size, or RNA yield. Values represent mean±standard error. * p<0.05.

FIGS. 3A and 3B show plasma exosomal RNA levels. In particular, FIG. 3A is a volcano plot of differential microRNA expression, showing the fold change vs. p-value for microRNA sequences identified by short non-coding RNA sequencing of plasma EVs in TBI patients with altered consciousness (GCS≤14) vs. TBI patients and healthy controls with normal consciousness (GCS=15). Sequences trending to high differential expression (log 2 Fold Change>0.07 and p<0.1) are shown in open circles. FIG. 3B is a graph plotting the relative frequency of miRNA vs. other RNA species in plasma exosomal RNA.

### DETAILED DESCRIPTION

Severe TBI affects more than 200,000 individuals annually in the United States and is a leading cause of mortality in men under 35 years (Korley et al., J Head Trauma Rehabil 2016, 31:379-387). The symptoms/effects of TBI can range from mild to severe, and may include loss of consciousness, mild to severe headache, nausea or vomiting, dilation of pupils, loss of coordination, sleeping disorders, convulsion, dizziness or fatigue, loss of balance, sensory symptoms, blurred vision, double vision, ringing sound in ears, loss of ability to smell, sensitivity to light or sound, mental symptoms, memory problems, alexia, speaking disorder, dysgraphia, mood swings, depression or anxiety, and/or coma. Mild TBI is under-reported, but may be as much as ten times more common than severe TBI, and is increasingly recognized as a contributor to chronic neurological dysfunction (McMahon et al., J Neurotrauma 2014, 31:26-33; and Seabury et al., JAMA Netw Open 2018, 1: e180210). Plasma biomarkers in TBI have generated excitement, with identification of multiple brain-specific molecules (e.g., GFAP, UCH-L1, NF-1, and tau) in the blood of TBI patients, and with FDA approval of a device (Brain Trauma Indicator) for rapid measurement of GFAP and UCH-LI levels in plasma (Korley et al., J Neurotrauma 2018, 36 (1) doi: 10.1089/neu.2017.5623). These biomarkers can be used to help clarify TBI diagnosis and prognosis, particularly in mild TBI.

EVs are membrane-bound particles released by the majority of human cells, including cells within the central nervous system (Heijnen et al., Blood 1999, 94:3791-3799; and Kowal et al., Curr Opin Cell Biol 2014, 29:116-125). EVs contain DNA, messenger RNA (mRNA), non-coding RNA, and proteins that are released into the microenvironment, and play key roles in cell-to-cell communication between neurons, glia and endothelial cells for complex functions including moderation of the blood-brain barrier (Li et al., RNA Biol 2013, 10:1333-1344; Proia et al., Int J Mol Med 2008, 21:63-67; and Schiera et al., J Cell Mol Med 2007, 11:1384-1394). EVs are a heterogeneous group that includes microvesicles (>100 nm) and smaller exosomes (<100 nm) that are generated through distinct cellular pathways and have distinct cargo. EVs have generated significant interest in neuro-oncology, as tumor-derived EVs shape the microenvironment by down-regulating the immune response and upregulating angiogenic factors, among other complex actions (Li et al., supra; Prioa et al., supra; Schiera et al., supra; and Mathivanan et al., Mol Cell Proteomics 2010, 9:197-208).

EVs have not been extensively studied in TBI, although one study identified a higher number of EVs in cerebrospinal fluid (CSF) of two subjects with severe (GCS 3-8) TBI compared to normal controls, and further isolated both GFAP and UCHL-1 in higher quantities in the TBI subjects compared to controls (Manek et al., Mol Neurobiol 2018, 55:6112-6128).

EVs in peripheral blood provide a lipid-membrane bound container of DNA, RNA, and proteins that are protected from circulating degradation enzymes, and therefore may provide a snapshot of the microenvironment in specific organ systems shortly before the peripheral blood sample is taken. In the studies described herein, plasma samples from TBI patients and healthy controls were used to assess plasma EVs as a biomarker source in TBI. These studies demonstrated the feasibility of isolating EVs, characterized EV size and frequency, and revealed the presence of both established (GFAP) and novel (short non-coding RNA) TBI biomarkers within EVs.

This document provides methods and materials for identifying and/or treating patients who have, or who are at an increased likelihood of having, a TBI, or mammals having, or being at an increased likelihood of having, a severe TBI as compared to a mild TBI. For example, this document provides methods and materials for identifying a mammal (e.g., a human) as having a TBI (e.g., a severe TBI or a mild TBI). Any appropriate mammal can be identified as having a TBI as described herein. For example, humans and other primates such as monkeys having can be identified as having a TBI as described herein. In some cases, dogs, cats, horses, cows, pigs, sheep, mice, or rats can be identified as having a TBI as described herein.

As described herein, a mammal (e.g., a human) can be identified as having (or as being likely to have) a TBI by detecting the presence of EVs with altered levels of one or more markers. Examples of markers that can be evaluated and used to classify a mammal (e.g., a human) as having or not having a TBI (e.g., a severe TBI) include, without limitation, miRNAs such as hsa-miR-1-3p (SEQ ID NO:2), hsa-miR-143-3p (SEQ ID NO: 3), hsa-miR-151b (SEQ ID NO:4), hsa-miR-27a-3p (SEQ ID NO:5), hsa-miR-29a-3p (SEQ ID NO:6), hsa-miR-328-3p (SEQ ID NO:7), hsa-miR-9-3p (SEQ ID NO:8), hsa-miR-99a-5p (SEQ ID NO:9), hsa-miR-99b-5p (SEQ ID NO:10), hsa-miR-155 -5p (SEQ ID NO: 11), and hsa-miR-30c-5p (SEQ ID NO:12).

In some embodiments, an miRNA marker provided herein can have a nucleotide sequence that is at least 90% (e.g., at least 91%, 92%, 93%, 94%, 95%, 96%, 97%, 98%, or 99%) identical to a sequence as set forth in any of SEQ ID NOs: 2-12. The percent sequence identity between a particular nucleic acid or amino acid sequence and a sequence referenced by a particular sequence identification number is determined as follows. First, a nucleic acid or amino acid sequence is compared to the sequence set forth in a particular sequence identification number using the BLAST 2 Sequences (B12seq) program from the stand-alone version of BLASTZ containing BLASTN version 2.0.14 and BLASTP version 2.0.14. This stand-alone version of BLASTZ can be obtained online at fr.com/blast or at ncbi.nlm.nih.gov. Instructions explaining how to use the B12seq program can be found in the readme file accompanying BLASTZ. B12seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seql.txt); -j is set to a file containing the second nucleic acid sequence to be compared (e.g., C:\seq2.txt) ; -p is set to blastn; -o is set to any desired file name (e.g., C:\output.txt); -q is set to -1; -r is set to 2; and all other options are left at their default setting. For example, the following command can be used to generate an output file containing a comparison between two sequences: C:\B12seq -i:\seql.txt -j c:\seq2.txt -p blastn -o c:\output.txt -q -1-r 2. To compare two amino acid sequences, the options of B12seq are set as follows: -i is set to a file containing the first amino acid sequence to be compared (e.g., C:\seql.txt); -j is set to a file containing the second amino acid sequence to be compared (e.g., C:\seq2.txt) ; -p is set to blastp; -o is set to any desired file name (e.g., C:\output.txt); and all other options are left at their default setting. For example, the following command can be used to generate an output file containing a comparison between two amino acid sequences: C:\B12seq -i c:\seql.txt-j c:\seq2.txt -p blastp -o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide residue is presented in both sequences. The percent sequence identity is determined by dividing the number of matches either by the length of the sequence set forth in the identified sequence or by an articulated length (e.g., 100 consecutive bases from a sequence set forth in an identified sequence), followed by multiplying the resulting value by 100. For example, the reference human miRNA sequence set forth in SEQ ID NO:2 is 22 nucleotides in length. A nucleotide sequence that has 21 matches when aligned with the reference sequence is 95.5 percent identical to the reference sequence (i.e., 21/22×100=95.5). It is noted that the percent sequence identity value is rounded to the nearest tenth. For example, 75.11, 75.12, 75.13, and 75.14 are rounded down to 75.1, while 75.15, 75.16, 7.17, 75.18, and 7.19 are rounded up to 7.2. It also is noted that the length value will always be an integer.

EVs can be obtained from any appropriate biological sample (e.g., blood, plasma, serum, or cerebrospinal fluid) using any appropriate method (e.g., density gradient ultracentrifugation or microfluidics). Any appropriate method can be used to determine if a mammal (e.g., a human) has altered (e.g., elevated or reduced) levels of one or more of the miRNA markers described herein. The term “elevated level” or “increased level” as used herein with respect to a miRNA level refers to a level of miRNA present within or otherwise associated with EVs that is greater (e.g., at least 5, 10, 25, 35, 45, 50, 55, 65, 75, 80, 90, or 100 or more percent greater) than the median level of that miRNA within or otherwise associated with control EVs from a mammal that does not have a TBI. The term “reduced level” or “decreased level” as used herein with respect to miRNA level refers to a level of miRNA present within or otherwise associated with EVs that is less (e.g., at least 5, 10, 25, 35, 45, 50, 55, 65, 75, 80, 90, or 100 percent less) than the median level of that miRNA within or otherwise associated with control EVs from a mammal that does not have a TBI. Appropriate methods for identifying EVs as having an elevated or reduced level of one or more miRNA markers as described herein include, without limitation, RNA sequencing and subsequent expression profiling, DNA sequencing and profiling, and protein characterization and profiling.

Once a mammal (e.g., a human) is identified as having EVs with altered (e.g., elevated or reduced) levels of one or more of the miRNAs described herein, the mammal can be classified as having, or being likely to have, a TBI. For example, a human identified as having EVs with altered levels of one or more (e.g., two, three, four, five, six, seven, eight, nine, ten, or all eleven) miRNAs selected from hsa-miR-1-3p (SEQ ID NO: 2), hsa-miR-143-3p (SEQ ID NO:3), hsa-miR-151b (SEQ ID NO:4), hsa-miR-27a-3p (SEQ ID NO:5), hsa-miR-29a-3p (SEQ ID NO:6), hsa-miR-328-3p (SEQ ID NO:7), hsa-miR-9-3p (SEQ ID NO:8), hsa-miR-99a-5p (SEQ ID NO:9), hsa-miR-99b-5p (SEQ ID NO: 10), hsa-miR-155-5p (SEQ ID NO:11), and hsa-miR-30c-5p (SEQ ID NO:12) can be classified as having, or being likely to have, a TBI. In some cases, a mammal (e.g., a human) identified as having EVs that do not exhibit altered levels of one or more (e.g., two, three, four, five, six, seven, eight, nine, ten, or all eleven) miRNAs selected from hsa-miR-1-3p (SEQ ID NO:2), hsa-miR-143-3p (SEQ ID NO:3), hsa-miR-151b (SEQ ID NO: 4), hsa-miR-27a-3p (SEQ ID NO:5), hsa-miR-29a-3p (SEQ ID NO:6), hsa-miR-328-3p (SEQ ID NO:7), hsa-miR-9-3p (SEQ ID NO:8), hsa-miR-99a-5p (SEQ ID NO:9), hsa-miR-99b-5p (SEQ ID NO:10), hsa-miR-155-5p (SEQ ID NO:11), and hsa-miR-30c-5p (SEQ ID NO:12) can be classified as not having, or not being likely to have, a TBI.

Any combination of markers can be evaluated. When increased levels of two miRNA markers are used to identify a mammal as having a TBI, for example, the two miRNAs can be hsa-mir-1-3p and hsa-mir-143-3p, hsa-mir-1-3p and hsa-mir-151b, hsa-mir-1-3p and hsa-mir-27a-3p, hsa-mir-1-3p and hsa-mir-29a-3p, hsa-mir-1-3p and hsa-mir-328-3p, hsa-mir-1-3p and hsa-mir-9-3p, hsa-mir-1-3p and hsa-mir-99a-5p, hsa-mir-1-3p and hsa-mir-99b-5p, hsa-mir-1-3p and hsa-mir-155-5p, hsa-mir-1-3p and hsa-mir-30c-5p, hsa-mir-143-3p and hsa-mir-151b, hsa-mir-143-3p and hsa-mir-27a-3p, hsa-mir-143-3p and hsa-mir-29a-3p, hsa-mir-143-3p and hsa-mir-328-3p, hsa-mir-143-3p and hsa-mir-9-3p, hsa-mir-143-3p and hsa-mir-99a-5p, hsa-mir-143-3p and hsa-mir-99b-5p, hsa-mir-143-3p and hsa-mir-155-5p, hsa-mir-143-3p and hsa-mir-30c-5p, hsa-mir-151b and hsa-mir-27a-3p, hsa-mir-151b and hsa-mir-29a-3p, hsa-mir-151b, and hsa-mir-328-3p, hsa-mir-151b and hsa-mir-9-3p, hsa-mir-151b and hsa-mir-99a-5p, hsa-mir-151b and hsa-mir-99b-5p, hsa-mir-151b and hsa-mir-155-5p, hsa-mir-151b and hsa-mir-30c-5p, hsa-mir-27a-3p and hsa-mir-29a-3p, hsa-mir-27a-3p and hsa-mir-328-3p, hsa-mir-27a-3p and hsa-mir-9-3p, hsa-mir-27a-3p and hsa-mir-99a-5p, hsa-mir-27a-3p and hsa-mir-99b-5p, hsa-mir-27a-3p and hsa-mir-155-5p, hsa-mir-27a-3p and hsa-mir-30c-5p, hsa-mir-29a-3p and hsa-mir-328-3p, hsa-mir-29a-3p and hsa-mir-9-3p, hsa-mir-29a-3p and hsa-mir-99a-5p, hsa-mir-29a-3p and hsa-mir-99b-5p, hsa-mir-29a-3p and hsa-mir-155-5p, hsa-mir-29a-3p and hsa-mir-30c-5p, hsa-mir-328-3p and hsa-mir-9-3p, hsa-mir-328-3p and hsa-mir-99a-5p, hsa-mir-328-3p and hsa-mir-99b-5p, hsa-mir-328-3p and hsa-mir-155-5p, hsa-mir-328-3p and hsa-mir-30c-5p, hsa-mir-9-3p and hsa-mir-99a-5p, hsa-mir-9-3p and hsa-mir-99b-5p, hsa-mir-9-3p and hsa-mir-155-5p, hsa-mir-9-3p and hsa-mir-30c-5p, hsa-mir-99a-5p and hsa-mir-99b-5p, hsa-mir-99a-5p and hsa-mir-155-5p, hsa-mir-99a-5p and hsa-mir-30c-5p, hsa-mir-99b-5p and hsa-mir-155-5p, hsa-mir-99b-5p and hsa-mir-30c-5p, or hsa-mir-155-5p and hsa-mir-30c-5p. Similarly, any combination of three, four, five, six, seven, eight, nine, ten, or all eleven of the aforementioned markers can be evaluated to determine whether a mammal has, or is likely to have, a TBI (e.g., a severe TBI).

As described herein, this document also provides methods and materials for treating a mammal identified as having, or as being likely to have, a TBI. Any appropriate mammal identified as having, or as being likely to have, a TBI can be treated by medical management such as, without limitation, administering one or more medications targeted to managing blood pressure, one or more medications targeted to managing cerebral perfusion pressure (e.g., vasopressors or antihypertensives), one or more medications targeted to managing intracranial pressure (e.g., mannitol or hypertonic saline), other medical interventions (e.g., one or more antibiotics to treat secondary infections), surgical treatment for removal of a mass or lesion or for decompression of the brain via direct craniectomy and bone removal, or any combination thereof. Having the ability to identify mammals who have or are likely to have a TBI can allow clinicians and patients to proceed with treatment options that mitigate the risk of longer term effects that can affect mammals with TBI. In addition, mammals identified as having, or being likely to have, a TBI can undergo more regular surveillance via, for example, examination and/or imaging studies (e.g., CT scans, MR images, or ultrasound-based techniques such as trans cranial Doppler studies) or invasive monitoring techniques through the use of an intracranial pressure monitor, a brain tissue oxygenation monitor, and/or an external ventricular drain, to detect changes in brain function early in its course.

The invention will be further described in the following example, which does not limit the scope of the invention described in the claims.

### EXAMPLES

### Example 1—Plasma Extracellular Vesicles as a Source of Biomarkers in TBI

### Materials and Methods

Subjects: Subject plasma samples (about 500 μL each) were selected from the TRACK-TBI biorepository from the University of Pittsburgh (Okonkwo et al., J Neurotrauma 2013, 30:1490-1497). A total of 20 plasma samples were obtained, including 15 samples taken after TBI and 5 samples taken from healthy controls. Subjects with complicated mild (GCS 13-15 with evidence of injury on CT scan), moderate (GCS 9-12), and severe (GCS 3-8) TBI were included. Attempts were made to select subjects with a wide range of injury mechanisms accompanying all severities of TBI, as well as from uninjured, healthy controls. Demographic and injury information are listed in

### Table 1

Isolation of EVs, western blot, and GFAP analysis: EVs within plasma were isolated using serial density gradient ultracentrifugation per a protocol described elsewhere (Cumba Garcia et al., Frontiers Oncol 2019, doi.org/10.3389/fonc.2019.00651). Concentrated EVs were then analyzed via nanotracker particle analysis using NanoSight (Malvern, NanoSight NS300). NanoSight allows for characterization of EVs, including both size and concentration, within a given sample. For western blot analysis, proteins were separated by electrophoresis on 4-20% SDS-PAGE. Following membrane transfer, the proteins were probed using the following antibodies: anti-Alix (Cell Signaling, #2171S, mouse monoclonal), anti-CD9 (Cell Signaling, #13174S, rabbit monoclonal), anti-CD63 (Santa Cruz Biotechnology, #sc-5275, mouse monoclonal) and anti-HSP90 (Cell Signaling, #4874S, rabbit polyclonal). The secondary antibody was horseradish peroxidase-conjugated goat anti-rabbit or goat anti-mouse (Jackson ImmunoResearch, #115-035-003, 111-035-003). Detection was by enhanced chemiluminescence (Thermo Fisher Scientific, #32106). For GFAP analysis, EVs were sonicated to break their membranes. GFAP expression was then tested in all samples via ELISA using a commercially available GFAP kit (abcam, cat #ab223867; Cambridge, MA) per the manufacturer's instructions.

Micro RNA analysis: Short non-coding RNA was isolated from samples using the miRNAeasy kit and QiaCube (Qiagen, Hilden, Germany). Preliminary quality assessment was performed using Nanodrop (Thermo-Fisher, Waltham, MA). Samples then underwent next generation sequencing using the Ilumina MiSeq 2000 with NebNext multiplex oligos for Illumina adapters (the adapter sequence was AGATCGGAAGAGCACACGTCT; SEQ ID NO:1). Analysis of short non-coding RNA sequencing was performed in collaboration with the Bioinformatics Core of the Mayo Clinic Center for Individualized Medicine. Sequencing data were analyzed using the OASIS-2.0 platform with the reference genome HG38. This investigated 28,263 unique RNA entities covering miRNA, piRNA, snoRNA, snRNA, and rRNA species. Differential expression analysis was used to identify differences in miRNA expression between groups.

Statistical Analysis: Descriptive statistics with medians, interquartile range, and proportions were used to describe continuous and categorical variables, respectively. Biomarker levels were treated as continuous data. The Mann-Whitney U test was used to assess for between-group differences for continuous variables. All statistical analysis was performed with JMP software (JMP Cary, NC).

### Results

Extracellular vesicles were isolated from 15 TBI subjects, including six CT-positive, mild TBI (GCS 13-15), three CT-positive, moderate TBI (GCS 9-12), and six CT-positive, severe TBI (GCS 3-8), as well as five healthy control subjects with no history of TBI or neurodegeneration. Given the relatively small sample size, subjects were divided into those who presented with an altered level of consciousness (GCS≤14) and those with a normal level of consciousness (GCS=15 and healthy controls) Demographic and injury information are listed in TABLE 1.

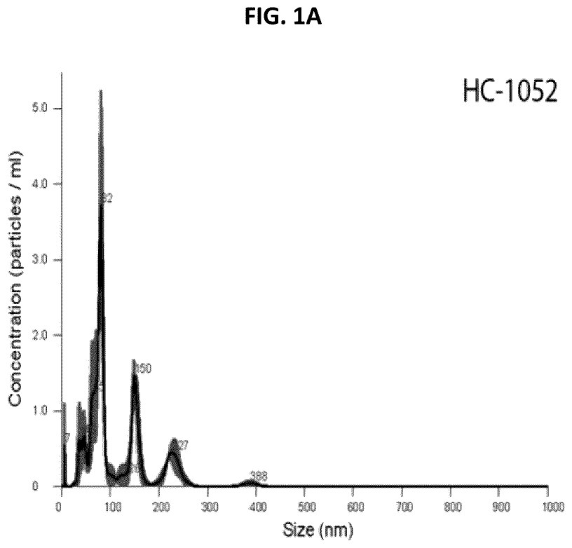

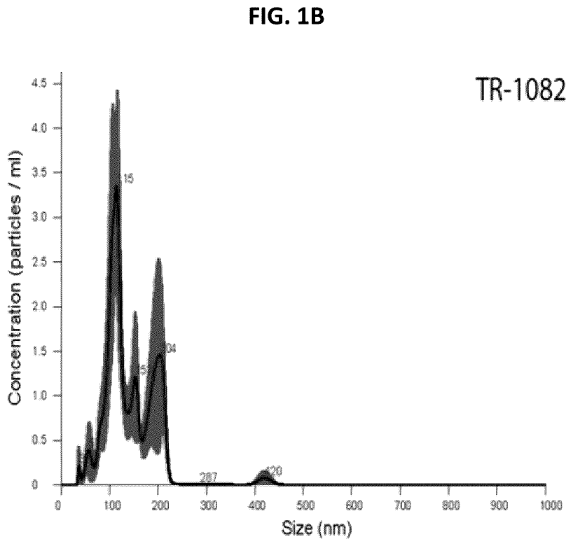

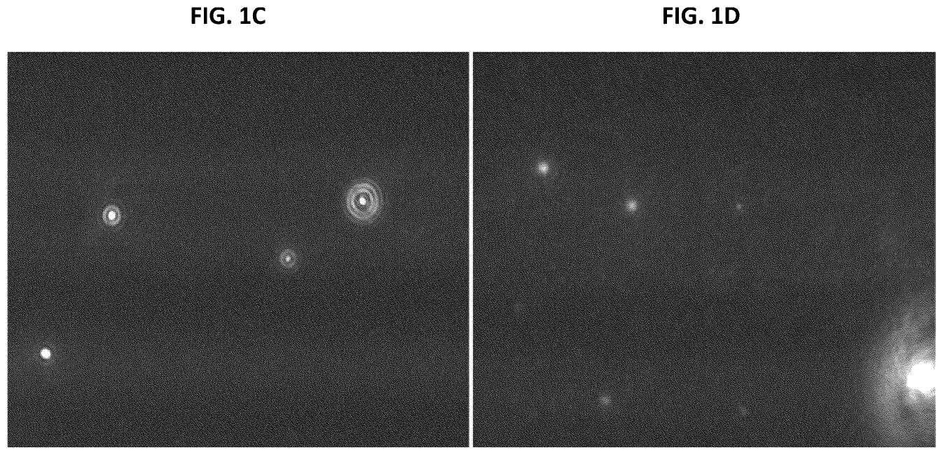

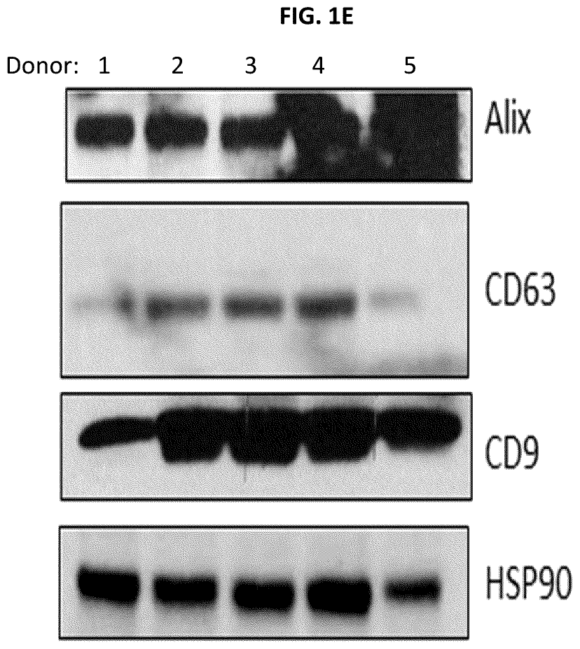

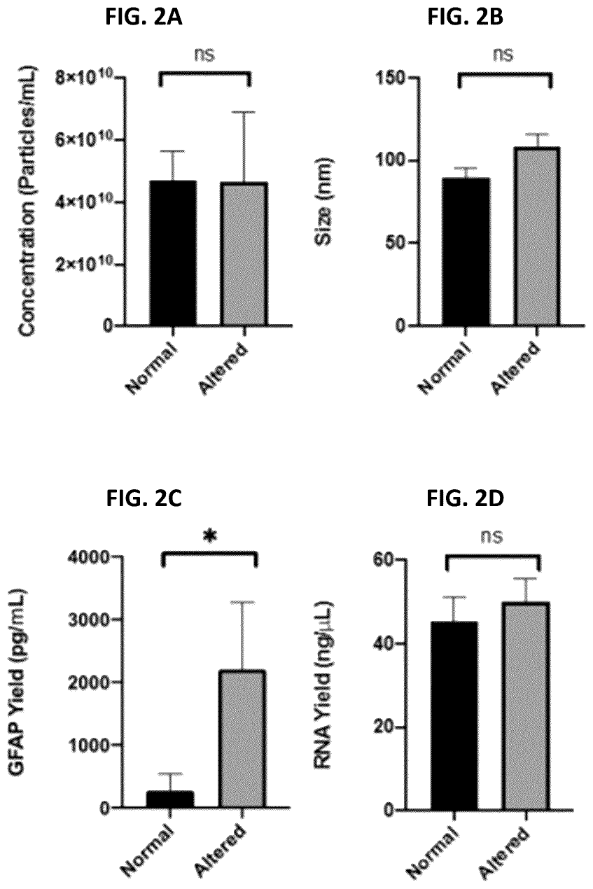

Isolation of EVs and GFAP analysis: EVs were successfully isolated in all samples analyzed. EV size and concentration were available for each sample. Data for a representative healthy control and a representative TBI subject are provided in the histograms of FIGS. 1A and 1B, respectively, and in the photomicrographs of FIGS. 1C and 1D, respectively. The histograms for both the healthy control (FIG. 1A) and the TBI patient (FIG. 1B) showed a substantial exosome peak (<100 nm) and multiple smaller microvesicle peaks (>100 nm). Common EV-associated proteins were assessed by Western blotting (FIG. 1E). EV size, concentration, GFAP concentration, and RNA yield for the entire cohort are shown in TABLE 2. There were no significant differences in EV size, concentration, or RNA yield between normal controls/TBI patients with normal consciousness (GCS=15) and TBI patients with altered consciousness (GCS≤14) (FIGS. 2A, 2B, and 2D). In contrast, analysis of GFAP yield by ELISA demonstrated significantly higher GFAP concentration in subjects with altered level of consciousness (GCS≤14) as compared to those with normal GCS (mean±standard error, GFAP concentration 2204.2 pg/mL±1067.2 pg/mL vs. 207.8 pg/mL±270.8 pg/mL, p=0.05; FIG. 2C).

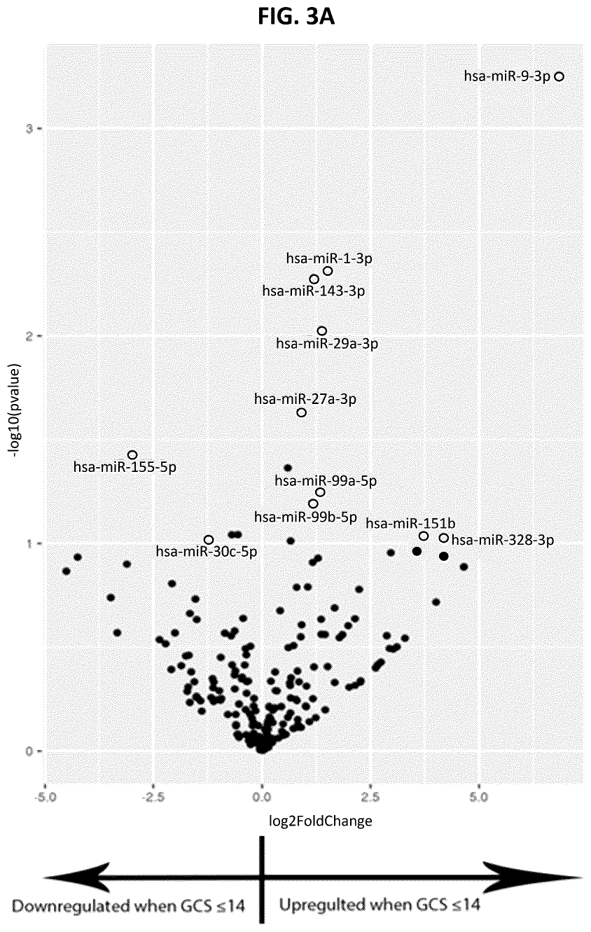

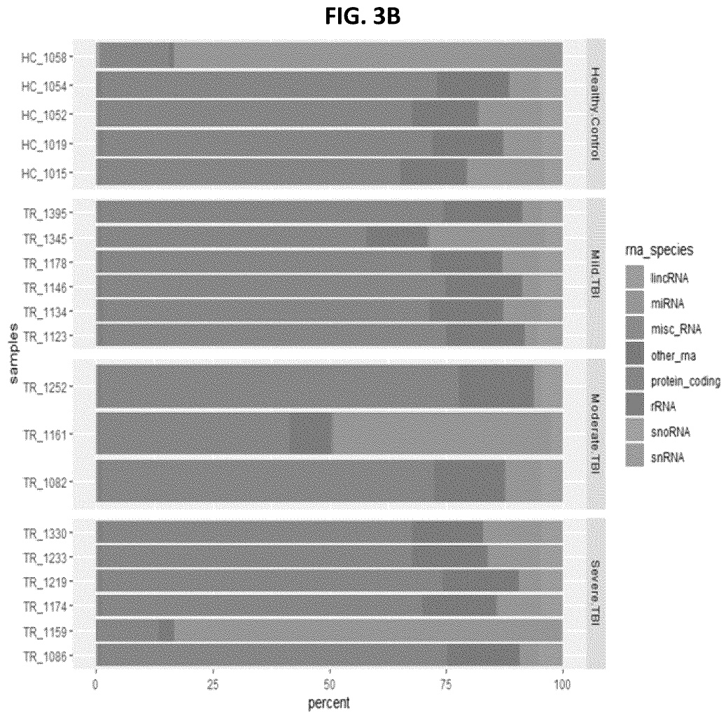

Short Non-Coding RNA Sequencing: RNA was successfully isolated from all samples, and RNA yields from the isolated EVs are listed in TABLE 2. Short non-coding RNA sequencing for plasma EVs from TBI patients and healthy controls were analyzed on the Illumina HiSeq 2500 platform and processed via a CAP-miRSeq pipeline. This allowed for unbiased identification of known and novel short non-coding RNA species beyond simply miRNAs. Differential expression analysis was performed for micro RNA sequences identified with thresholds of log 2 fold change [0.7] and a p-value<0.1 given the low number of samples with widely varying demographics, injury mechanism, and presentation GCS. Using these criteria, trends for nine highly upregulated miRNAs and two highly downregulated miRNAs were identified in TBI patients with an altered level of consciousness as compared to TBI patients and healthy controls with a normal level of consciousness (FIG. 3A, TABLES 3A and 3B). These reached statistical significance (p≤0.05) for 7 of the 11 individual miRNA sequences identified. Further, Ingenuity pathway analysis of highly differentially expressed miRNAs among all TBI patients relative to controls identified top disease and disorder pathways targeted, including cancer, gastrointestinal disease, and organismal injuries/abnormalities, while the top molecular and cellular pathways that were identified included cell death/survival, cell-to-cell signaling, and cellular assembly/organization (TABLE 4).

microRNAs have been well characterized in existing data-bases, which facilitated their analysis for the present study. However, microRNAs actually made up a minority of

RNA species found within plasma EVs (FIG. 3B). The majority of the RNA found was actually protein-coding mRNA. Due to the specific parameters of the sequencing paradigm, which was aimed at short non-coding RNA, the protein-coding transcripts found in the present studies were not meaningfully quantified. Nevertheless, they were present in abundance. Similarly, multiple additional species of non-coding RNA were found, including small nucleolar RNA (snoRNA), small nuclear RNA (snRNA), ribosomal (rRNA), and long intergenic noncoding RNA (lincRNA).

Thus, the studies described herein demonstrated that EVs can be isolated from peripheral blood after TBI, and indicated that they contain brain-specific biomarkers that can be analyzed and may be useful in the diagnosis, prognosis, and treatment monitoring of TBI.

TABLE 1
Cohort selected demographics, injury
mechanism and presentation GCS

| | | | Injury | Presentation |
|---|---|---|---|---|
| TBI | Age | Sex | Mechanism | GCS |
| TR-1345 | 22 | Female | MVC | 15 |
| TR-1134 | 41 | Male | MVC | 15 |
| TR-1146 | 32 | Male | Fall | 15 |
| TR-1395 | 46 | Male | MVC | 14 |
| TR-1178 | 25 | Male | MVC | 14 |
| TR-1123 | 72 | Male | Fall | 13 |
| TR-1161 | 33 | Male | Assault | 11 |
| TR-1252 | 24 | Male | MVC | 10 |
| TR-1082 | 55 | Female | Fall | 9 |
| TR-1330 | 35 | Male | Other | 6T |
| TR-1174 | 59 | Male | MVC | 3 |
| TR-1086 | 56 | Female | Fall | 3 |
| TR-1219 | 45 | Male | MVC | 3 |
| TR-1233 | 30 | Male | MVC | 3 |
| TR-1159 | 23 | Male | MVC | 3 |
| Healthy Control | | | | |
| HC-1054 | 25 | Male | N/A | 15 |
| HC-1052 | 31 | Male | N/A | 15 |
| HC-1015 | 40 | Male | N/A | 15 |
| HC-1019 | 53 | Female | N/A | 15 |
| HC-1058 | 63 | Male | N/A | 15 |

MVC = Motor Vehicle Collision

TABLE 2
MV/E concentration, mean size, GFAP concentration and RNA concentration

| | [MV/E] (particles/mL) | MV/E Size (nm) | [GFAP] (pg/mL) | [RNA] (ng/uL) |
|---|---|---|---|---|
| TBI | | | | |
| TR-1345 (GCS 15) | 36,300,000,000 | 85.2 | 2166.68 | 61.4 |
| TR-1134 (GCS 15) | 93,900,000,000 | 68.6 | Undetectable | 37.1 |
| TR-1146 (GCS 15) | 145,000,000,000 | 89.5 | Undetectable | 30.7 |
| TR-1395 (GCS 14) | 12,100,000,000 | 107.1 | 3489.56 | 42.4 |
| TR-1178 (GCS 14) | 89,400,000,000 | 67.7 | 136.4 | 46 |
| TR-1123 (GCS 13) | 13,400,000,000 | 124.9 | 2164.03 | 57.6 |
| TR-1161 (GCS 11) | 282,000,000,000 | 71.7 | Undetectable | 33 |
| TR-1252 (GCS 10) | 18,700,000,000 | 137.2 | 466.6 | 54.7 |
| TR-1082 (GCS 9) | 20,100,000,000 | 139 | Undetectable | 62.4 |
| TR-1330 (GCS 6T) | 8,480,000,000 | 92.7 | 1381.7 | 47.6 |
| TR-1174 (GCS 3) | 11,600,000,000 | 154.1 | Undetectable | 76.8 |
| TR-1086 (GCS 3) | 32,200,000,000 | 105 | 221.99 | 32.3 |
| TR-1219 (GCS 3) | 28,400,000,000 | 85.1 | 3834.9 | 42.6 |
| TR-1233 (GCS 3) | 31,700,000,000 | 106.6 | 13122.5 | 86.6 |
| TR-1159 (GCS 3) | 11,300,000,000 | 108.3 | 1633.1 | 16 |
| Healthy Control | | | | |
| HC-1054 (GCS 15) | 64,200,000,000 | 64.6 | Undetectable | 55.9 |
| HC-1052 (GCS 15) | 12,200,000,000 | 114 | Undetectable | 65.3 |
| HC-1015 (GCS 15) | 45,800,000,000 | 114.8 | Undetectable | 40.2 |
| HC-1019 (GCS 15) | 53,400,000,000 | 79.1 | Undetectable | 53.2 |
| HC-1058 (GCS 15) | 55,200,000,000 | 97.8 | Undetectable | 17 |
| | Analysis | | | |
| | Mean [MV/E] (particles/mL) | Mean MV/E size (nm) | [GFAP] (pg/mL) | [RNA] (ng/uL) |
| Altered GCS | 4.7e10±2.2e10 | 108.3 ± 7.7 | 2204.2 ± 1067.2 | 49.8 ± 5.6 |
| Normal GCS | 6.3e10±1.4e10 | 89.2 ± 6.7 | 207.8 ± 270.8 | 45.1 ± 5.9 |
| | (NS) | (NS) | (p = 0.02) | (NS) |

TABLE 3A
Upregulated and downregulated miRNA sequences in subjects with
altered presentation GCS compared to normal presentation GCS

| Upregulated miRNA | Log2 Fold Change | p-value |
|---|---|---|
| hsa-miR-1-3p | 1.51 | p < 0.01 |
| hsa-miR-143-3p | 1.19 | p < 0.01 |
| hsa-miR-151b | 3.71 | p = 0.09 |
| hsa-miR-27a-3p | 0.89 | p = 0.02 |
| hsa-miR-29a-3p | 1.37 | p < 0.01 |
| hsa-miR-328-3p | 4.18 | p = 0.09 |
| hsa-miR-9-3p | 6.84 | p < 0.01 |
| hsa-miR-99a-5p | 1.32 | p = 0.05 |
| hsa-miR-99b-5p | 1.15 | p = 0.06 |
| Downregulated miRNA | | |
| hsa-miR-155-5p | −2.99 | p = 0.03 |
| hsa-miR-30c-5p | −1.24 | p = 0.09 |

TABLE 3B
miRNA sequences

| miRNA | Sequence | SEQ ID NO: |
|---|---|---|
| hsa-miR-1-3p | uggaauguaaagaaguauguau | 2 |
| hsa-miR-143-3p | ugagaugaagcacuguagcuc | 3 |
| hsa-miR-151b | ucgaggagcucacagucu | 4 |
| hsa-miR-27a-3p | uucacaguggcuaaguuccgc | 5 |
| hsa-miR-29a-3p | uagcaccaucugaaaucgguua | 6 |
| hsa-miR-328-3p | cuggcccucucugcccuuccgu | 7 |
| hsa-miR-9-3p | auaaagcuagauaaccgaaagu | 8 |
| hsa-miR-99a-5p | aacccguagauccgaucuugug | 9 |
| hsa-miR-99b-5p | cacccguagaaccgaccuugcg | 10 |
| hsa-miR-155-5p | uuaaugcuaaucgugauaggggu | 11 |
| hsa-miR-30c-5p | uguaaacauccuacacucucagc | 12 |

TABLE 4
Top pathways for diseases disorders or molecular
and cellular functions targeted by differentially
expressed plasma exosomal miRNAs, comparing
TBI patients with altered consciousness
(GCS < 14) and TBI patients/healthy
controls with normal consciousness (GCS ≥ 14).
Diseases and Disorders

| Name | p-value range | # Molecules |
|---|---|---|
| Cancer | 4.75E−02-1.35E−12 | 10 |
| Gastrointestinal Disease | 3.84E−02-1.35E−12 | 10 |
| Organismal Injury and | 4.78E−02-1.35E−12 | 10 |
| Abnormalities | | |
| Respiratory Disease | 8.49E−03-1.35E−12 | 8 |
| Reproductive System | 3.43E−02-1.00E−08 | 9 |
| Disease | | |

Molecular and Cellular Functions

| Name | p-value range | # Molecules |
|---|---|---|
| Cell Death and Survival | 4.13E−02-8.72E−04 | 6 |
| Cell-to-Cell Signaling | 1.97E−02-9.05E−04 | 4 |
| and Interaction | | |
| Cellular Assembly and | 2.82E−02-9.05E−04 | 4 |
| Organization | | |
| Cellular Development | 4.22E−02-9.05E−04 | 9 |
| Nervous System Devel- | 3.04E−02-9.05E−04 | 6 |
| opment and Function | | |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for clearing a mammal for normal activity, wherein said mammal had a TBI or is suspected of having a TBI, wherein said method comprises:
(a) identifying said mammal as having EVs that comprise levels of hsa-miR-1-3p (SEQ ID NO:2), hsa-miR-143-3p (SEQ ID NO:3), hsa-miR-151b (SEQ ID NO:4), hsa-miR-27a-3p (SEQ ID NO:5), hsa-miR-29a-3p (SEQ ID NO:6), hsa-miR-328-3p (SEQ ID NO: 7), hsa-miR-9-3p (SEQ ID NO:8), hsa-miR-99a-5p (SEQ ID NO:9), hsa-miR-99b-5p (SEQ ID NO:10), hsa-miR-155-5p (SEQ ID NO:11), and hsa-miR-30c-5p (SEQ ID NO: 12) that are less than 5 percent altered as compared to the levels of said miRNAs in EVs from a corresponding mammal that does not have a TBI, and
(b) instructing said mammal to resume one or more normal activities.

2. The method of claim 1, wherein said mammal is a human.

3. The method of claim 1, wherein said EVs are from a blood sample obtained from said mammal.

4. The method of claim 3, wherein said blood sample is a plasma sample.

5. The method of claim 1, wherein said method comprises instructing said mammal to resume a sporting activity.
